(19) United States
(12) Patent Application Publication
(10) Pub. No.: US 2005/0239201 A1
(43) Pub. Date: Oct. 27, 2005

(54) Methods of inducing differentiation of stem cells into a specific cell lineage

(75) Inventors: Richard Mollard, Brighton (AU); Alan Trounson, Ashburton (AU); Mark Denham, Dingley (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Monash University, Clayton (AU)

(21) Appl. No.: 10/507,765

(22) PCT Filed: Mar. 14, 2003
(86) PCT No.: PCT/AU03/00310

§ 371 Date: May 11, 2005

(30) Foreign Application Priority Data
Mar. 15, 2002 (AU) PS 1128

(51) Int. Cl. C12N 5/06; C12N 5/08

(52) U.S. Cl. 435/354; 435/366

(57) ABSTRACT

The present invention relates to methods of inducing differentiation of stem cells into a specific cell lineage, preferably lung cells. In particular, the invention relates to in vitro methods of inducing differentiation of stem cells into a specific cell lineage. The invention also relates to methods of producing and recovering differentiated stem cells of a specific cell lineage. The invention also includes differentiated stem cells and cell lineages produced by the methods of the present invention. In one aspect of the present invention there is provided a method of inducing differentiation of a stem cell into a specific cell lineage, preferably lung cells, the method including: culturing a stem cell in vitro in the presence of a tissue sample and/or extracellular medium of a tissue sample, under conditions that induce differentiation of the stem cell into a specific cell lineage, wherein the differentiated stem cell is the same cell type as the tissue sample.

The present invention relates to methods of inducing differentiation of stem cells into a specific cell lineage. In particular, the invention relates to in vitro methods of inducing differentiation of stem cells into a specific cell lineage. The invention also relates to methods of producing and recovering differentiated stem cells of a specific cell lineage. The invention also includes differentiated stem cells and cell lineages produced by the methods of the present invention.

### INTRODUCTION

Stem cells are undifferentiated cells which can give rise to a succession of mature functional cells. Embryonic stem (ES) cells are derived from the embryo and in the mouse, when maintained in vitro in the presence of leukocyte inhibitory factor (LIF) are pluripotent, thus possessing the capability of developing into any organ, cell type or tissue type. Furthermore, when grown in hanging drops as a cell aggregate in the absence of LIF, mouse ES cells differentiate into representatives of all three embryonic germ layers, namely endoderm, mesoderm and ectoderm. Such aggregates are thus called embryoid bodies (EBs).

The process of differentiation in stem cells involves selective development of immature cells to committed and fully mature cells of various cell lineages. Derivatives of such cell lineages include, respiratory, muscle, neural, skeletal, blood (hematopoietic), endothelial and epithelial cells. Differentiation of stem cells is known to be triggered by various growth factors and regulatory molecules. During differentiation the expression of stem cell specific genes and markers are often lost and cells acquire gene expression profiles of somatic cells or their precursors. In some cases, “master” genes have been described which control differentiation versus self-renewal.

Whilst differentiation of stem cells into various cell lineages may be induced with a degree of certainty, a differentiation outcome of a population of stem cells is less predictable. Placing the cells under conditions which induce specific cell types has been one form of an attempt to regulate the differentiation outcome. These conditions typically include growing the cells to high or low density, changing media, introducing or removing cytokines, hormones and growth factors, creating an environment which suits differentiation toward a specific cell type, such as providing a suitable substrate.

Generally, when a stem cell culture is induced to differentiate, the differentiated population is analyzed for particular cell types by expression of genes, markers or phenotypic analysis. The respective cell types are then typically selectively cultured to enrich their percentage population to eventually obtain a pure cell type and culture. However, recovering differentiated cells of a specific cell lineage in this manner is time-consuming and complicated.

The recovery of differentiated stem cells of a specific cell lineage can be useful for transplantation or drug screening and drug discovery in vitro and in vivo. Methods of inducing differentiation of stem cells and differentiated cells produced therefrom may be used for the study of cellular and molecular biology of tissue development, for the discovery of genes, proteins, such as differentiation factors that play a role in tissue development and regeneration.

In particular, the induction of stem cells to differentiate into a specific cell lineage is useful for transplantation and therapeutic purposes, as well as providing potential human disease models in culture (e.g. for testing pharmaceuticals). The induction of differentiation of stem cells into a specific cell lineage is especially useful in developing therapeutic methods and products for tissue specific diseases and conditions.

Therefore there remains a need for providing effective methods of inducing differentiation of stem cells in vitro into a specific cell lineage, and then preferably providing efficient and reliable methods of recovering differentiated stem cells of a specific cell lineage.

### SUMMARY OF THE INVENTION

In one aspect of the present invention there is provided a method of inducing differentiation of a stem cell into a specific cell lineage, the method including:
culturing a stem cell in vitro in the presence of a tissue sample and/or extracellular medium of a tissue sample said sample selected during organogenesis of the tissue, under conditions that induce differentiation of the stem cell into a specific cell lineage.

Preferably, the tissue sample is treated to form tissue cells in a substantially single cell suspension. Alternatively, the tissue sample is prepared as a sheet prior to culturing with the stem cells. The tissue cells are preferably derived from embryonic, foetal or post-partum tissue. Most preferably, the tissue cells are mesenchymal cells. The tissue cells are preferably derived from embryonic lung mesenchyme.

The stem cells used in the methods of the present invention are preferably embryonic stem (ES) cells. The tissue cells and/or the stem cells used in the methods of the present invention may be tagged. Preferably, the stem cells used express a transgenic marker protein that allows for identification of differentiated stem cells. The stem cells may be induced to differentiate into specific cell lineages, preferably selected from the group consisting of respiratory, pancreatic, mammary, renal, intestinal, and hepatic.

In another aspect of the present invention there is provided a method of inducing differentiation of a stem cell into a specific cell lineage, the method including the steps of:
mixing a first sample of stem cells with a second sample of tissue cells said cells selected during organogenesis of the tissue to form a cell mixture;
culturing the cell mixture in vitro, under conditions that induce differentiation of a stem cell into a specific cell lineage.

Preferably, the tissue cells are in a substantially single cell suspension prior to mixing with the stem cells. Alternatively, the tissue cells are prepared as a sheet for wrapping an undifferentiated embryoid body. Undifferentiated embryoid bodies are preferably prepared by cultivating ES cells in hanging drops in the presence of LIF. Preferably, the culturing step includes allowing the cell mixture to grow on a permeable membrane, wherein the membrane is in contact with a culture medium, such that the stem cells are induced to differentiate into a specific cell lineage.

In another aspect of the present invention there is provided a method of producing differentiated stem cells of a specific cell lineage, the method including:
culturing stem cells in vitro in the presence of a tissue sample and/or extracellular medium of a tissue sample, said [symbol] organogenesis [symbol] stem cell into a specific cell lineage; and
recovering differentiated stem cells of a specific cell lineage.

Preferably, the tissue sample is treated to form tissue cells in a substantially single cell suspension prior to culturing with the stem cell. Alternatively, the tissue cells are prepared as a sheet for wrapping an undifferentiated embryoid body.

In a preferred aspect of the present invention there is provided a method of producing differentiated stem cells of a specific cell lineage, the method including:
culturing stem cells in vitro in the presence of tissue cells, said cells selected during organogenesis of the tissue, under conditions that induce differentiation of a stem cell into a specific cell lineage; and
recovering the differentiated stem cells of a specific cell lineage.

Preferably, the tissue cells are in a substantially single cell suspension prior to culturing with the stem cells. Alternatively, the tissue cells are prepared as a sheet for wrapping an undifferentiated embryoid body.

The culturing step preferably includes allowing the stem cells to grow on a first surface of a permeable membrane and allowing the tissue cells to grow on an opposite second surface of the permeable membrane, wherein the membrane is in contact with a culture medium, such that the stem cells are induced to differentiate into a specific cell lineage. Differentiated stem cells of a specific cell lineage may then be recovered from the first surface of the permeable membrane.

In the methods of the present invention, the tissue cells may be derived from embryonic, foetal or post-partum tissue. Preferably, the tissue cells are embryonic mesenchymal cells. More preferably, the tissue cells are derived from lung mesenchyme tissue, more preferably embryonic lung mesenchyme. The stem cells used in the methods of the present invention are preferably embryonic stem (ES) cells. The tissue cells and/or the stem cells used in the methods of the present invention may be tagged. Preferably, the stem cells used express a transgenic marker protein that allows for identification of differentiated stem cells. The stem cells may be induced to differentiate into specific cell lineages, preferably selected from the group consisting of respiratory, pancreatic, mammary, renal, intestinal, and hepatic.

The types of cell lineages will be determined by the tissues that are co-cultured with the stem cells or embryonic stem cells. The tissues and cell lineages are from tissues that can undergo organogenesis such as, but not limited to, respiratory, pancreatic, mammary, renal, intestinal or hepatic tissues.

In the methods of the present invention, the culturing step may preferably include the addition of a growth factor to enhance stem cell differentiation. Suitable growth factors may be preferably selected from epidermal growth factor (EGF), hepatocyte growth factor (HGF) and fibroblast growth factors (FGFs) or steroid hormones (for example, glucocorticoids, vitamin A, thyroid hormone, androgens and estrogens).

In yet another aspect of the invention, there is provided differentiated stem cells of a specific cell lineage produced according to the methods as hereinbefore described. Preferably, the differentiated stem cell is a lung, kidney, cardiomyocyte, mammary cell, salivary cell, , hepatic cell, intestinal cell or pancreatic cells. The present invention also provides differentiated stem cells produced according to the methods of the invention that may be used for tissue repair, transplantation, cell therapy or gene therapy.

The present invention further provides a cell composition including a differentiated stem cell produced by the methods of the present invention, and a carrier.

### FIGURES

FIG. 1 Mouse ES cell/respiratory tissue aggregates. Double b-galactoside staining (blue stain) and surfactant C immunohistochemistry (brown stain) demonstrates that the ES cell derivatives are induced to form bronchiolar duct-like structures that are immunoreactive to the respiratory specific marker (A-D). Note that after 6 days in culture (A and B), surfactant C immunoreactivity can be observed throughout the entire bronchiolar-like duct throughout the ES cell derivative cytoplasm. After twelve days in culture, surfactant C immunoreactivity is restricted to a sub-set of the bronchiolar-like duct population and within these ducts, to the cell surface of the ES cell derivatives (see red arrows in C and D).

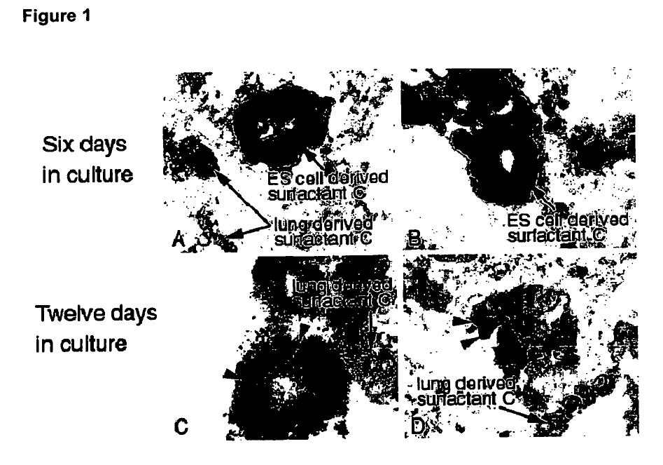

FIG. 2 Human embryonic stem (hES) cell directed surfactant C (Sp-C) expression. hEScell/mouse lung aggregates were grown for 6 days in vitro. Cell nuclei are identified by the generic nuclear stain Hoechst 33342 (blue), hES cell derivatives are identified by green fluorescence, and Sp-C localisation by an anti- mouse and human Sp-C specific antibody (red). Sp-C localisation is observed within the mouse tissue and within hES derivatives.

FIG. 3 shows fifty neurospheres following plating in 35 mm dishes. (A) control media and (B) HGF+ containing media (HGF+ media is DMEM containing 3% charcoal stripped foetal calf serum, 10 μg/ml insulin, 1 μg/ml cholera toxin, 25 ng/ml epidermal growth factor, 10 ng/ml hepatocyte growth factor and 25 ng/ml FGF7)—note foci. (C) Double Hoechst (blue) and anti-surfactant C (red) fluorescence to reveal respiratory differentiation. (D) High power (hp) magnification of (C) to highlight double labelling of single cells.

FIG. 4 shows reverse transcriptase—polymerase chain reaction (RT-PCR) for endodermal and respiratory markers of mouse embryoid bodies and neurospheres cultured for 8 days in DMEM+10% FCS and each of the indicated growth factor supplements.

### DETAILED DESCRIPTION OF THE INVENTION

In one aspect of the present invention there is provided a method of inducing differentiation of a stem cell into a specific cell lineage, the method including:
culturing a stem cell in vitro In the presence of a tissue sample and/or extracellular medium of a tissue sample said sample selected during organogenesis of the tissue, under conditions that induce differentiation of the stem cell into a specific cell lineage.

Applicants have found that culturing stem cells in the presence of a tissue sample of a specific cell type provides an effective means of producing differentiated stem cells reminiscent of a specific cell lineage. They have further found that the tissue is of the type that can undergo organogenesis. It is at this time that the tissue is best used to induce differentiation of stem cells. In the methods of the present invention the differentiation outcome of a stem cell can be determined, as the differentiated stem cells are the same cell type (ie preferably express a similar set of markers) as the tissue sample used in co-culture with the stem cells.

In the methods of the present invention the tissue sample is preferably treated to form tissue cells in a substantially single cell suspension prior to culturing with the stem cell. Tissue cells in a substantially single cell suspension enhance the exposure and contact of secreted products and chemical cues produced by the tissue cells to act on and induce differentiation of a stem cell in co-culture. The applicants have found that tissue cells in single cell suspension that are co-cultured with stem cells tend to form heterotypic tissue that comprise differentiated stem cells aggregated with the tissue cells, wherein the differentiated stem cells are the same cell type as the tissue cells. Furthermore, when tissue cells are prepared as a sheet in which an undifferentiated embryoid body is wrapped, the applicants have found that the stem cells will form a heterotypic tissue comprised of cells characteristic of the tissue from which the tissue sheet was derived.

The phrase “inducing differentiation of a stem cell into a specific cell lineage” as used herein is taken to mean causing a stem cell to develop into a specific differentiated cell lineage as a result of a direct or intentional influence on the stem cell. Influencing factors that may induce differentiation in a stem cell can include cellular parameters such as ion influx, a pH change and/or extracellular factors, such as secreted proteins, such as but not limited to growth factors and cytokines that regulate and trigger differentiation. It may include culturing the cell to confluence and may be influenced by cell density.

In a preferred embodiment of the invention differentiation of a stem cell into a specific cell lineage is achieved by co-culturing tissue cells in a substantially single cell suspension with stem cells to preferably form heterotypic tissue (ie differentiated stem cells aggregated with tissue cells). Heterotypic recombinations of differentiated stem cells aggregated with the tissue cells are preferably formed, wherein the differentiated stem cells are the same cell type as the tissue cells. Tissue cells that are in a substantially single cell suspension allow for enhanced induction of stem cells to differentiate and to form heterotypic re-association in vitro with the tissue cells.

The term “specific cell lineage” as used herein is taken to refer to the ancestry of a particular cell type, including ancestral cells and all of the subsequent cell divisions which occurred to produce the specific cell type. Differentiated stem cells of a specific cell lineage are a group of cells that have the same cell type. Cells of the same cell type are similar to each other, along with their associated intercellular substances, and perform the same function within a multicellular organism. Cells of the same cell type preferably express a similar set of markers. Major tissue cell types include, but are not limited to, epithelial, endothelial connective, skeletal, muscular, glandular, and nervous tissues. In the present methods, the stem cells are preferably co-cultured with tissue cells such that the stem cells are induced to differentiate into a specific cell lineage that is the same cell type as the tissue cells.

In the methods of the present invention a stem cell is undifferentiated prior to culturing and is any cell capable of undergoing differentiation. The stem cell may be selected from the group including, but not limited to, embryonic stem cells, pluripotent stem cells, haematopoietic stem cells, totipotent stem cells, mesenchymal stem cells, neural stem cells, or adult stem cells. The stem cells are preferably derived from a mammalian animal, most preferably, but not limited to, a mouse or human.

The stem cells used in the methods of the present invention are preferably embryonic stem (ES) cells. The stem cell is preferably a mammalian embryonic stem cell which may be derived directly from an embryo, from a culture of embryonic stem cells, or from somatic nuclear transfer. Whilst, the stem cell may be derived from other mammalian animals, they are most preferably human embryonic stem cells. The embryonic stem (ES) cell used in the present method includes an embryonic cell derived from an embryo or a cell derived from extraembryonic tissue. Suitable embryonic stem cells include those that are commercially available such as those previously described (Reubinoff et al., 2000) or hES1, hES3, hES4, hES5, or hES6. These cells may be obtained from ES Cell International Pte Ltd.

The term “embryo” as used herein is defined as any stage after fertilisation which can be up to 2 weeks post conception in mammals. The embryonic period for mammals, such as a mouse is approximately 4-6 days. An embryo develops from repeated division of cells and includes the stages of a blastocyst stage which comprises an outer trophectoderm and an inner cell mass (ICM). The embryo may be an in vitro fertilised embryo or it may be an embryo derived by transfer of a somatic cell or cell nucleus into an enucleated oocyte preferably of human or non-human origin. Extraembryonic tissue includes cells produced by the embryo that make up the placenta.

Suitable embryonic stem (ES) cells that may be used in the methods of the present invention may include mammalian ES cells. ES cells are known to have pluripotent properties and may be induced to undergo controlled differentiation to produce diverse cell lineages in vitro.

The stem cells may be cultured in the presence of tissue cells to induce differentiation of the stem cells into a specific cell lineage. The embryonic stem cells may be cultured in either methyl cellulose containing media in bacterial grade petri dishes or hanging drops to prevent their adherence to the surface of the culture dish, thus inducing the generation of colonies of differentiated cells known as embryoid bodies (EBs). EBs contain cellular representatives of all three embryonic germ layers (ectoderm, mesoderm and endoderm) and under specific culture conditions may be instructed and manipulated to generate pure preparations of specific cell lineages.

The stem cells used in the present methods may be derived from an embryonic cell line, embryonic tissue, or somatic nuclear transfer. The embryonic stem cells may be cells which have been cultured and maintained in an undifferentiated state. The ES cells used may be either as a single cell suspension if intended for culture with a single cell suspension of tissue sample. Alternatively, the ES cells may be grown as hanging drops in the presence of LIF such that they may form undifferentiated aggregates if intended for culture wrapped in a prepared tissue sheet. These aggregates are known as undifferentiated embryoid bodies.

The stem cells suitable for use in the present methods may be derived from a patient's own tissue. This would enhance compatibility of differentiated tissue grafts derived from the stem cells with the patient. The stem cells may be genetically modified prior to use through introduction of genes that may control their state of differentiation prior to, during or after their exposure to the embryonic cell or extracellular medium from an embryonic cell. They may be genetically modified through introduction of vectors expressing a selectable marker under the control of a stem cell specific promoter such as Oct-4.

The stem cells may be genetically modified at any stage with markers so that the markers are carried through to any stage of cultivation. The markers may be used to purify the differentiated or undifferentiated stem cell populations at any stage of cultivation. Transgenic markers, for example, green fluorescent protein (GFP) allows for isolation of pure stem cell derivatives utilising fluorescence activated sorting (FACs) at required lengths of time following induction. Differentiated stem cells produced by the methods of the present invention may be genetically modified to bear mutations. Genetically modified stem cells that are induced to differentiate to specific cell lineages may be useful culture models and may provide a route for delivery of gene therapy.

In the methods of the present invention the stem cell can be induced to differentiate into a specific cell lineage, preferably selected from the group consisting of respiratory, pancreatic, mammary, renal, intestinal, hepatic, haematopoietic, muscle or cardiac cell lineages. Preferably, the stem cell is induced to differentiate into a respiratory cell lineage.

The term “tissue sample” as used herein is taken to include, but not be limited to, tissue extracts, cell culture medium, biopsy specimens or resected tissue. The tissue sample, preferably includes tissue cells. A tissue sample, preferably includes tissue cells, that are a group of cells similar to each other, along with their associated intercellular substances, which perform the same function within a multicellular organism. Major tissue cell types include, but are not limited to, epithelial, endothelial connective, skeletal, muscular, glandular, and nervous tissues.

The tissue sample is preferably derived from a mammalian organism, most preferably a human subject. More preferably, the tissue sample is, but not limited to, tissue derived from various mammalian organs, such as, respiratory, reproductive, kidney, brain, heart, muscle and skeletal. The tissue sample preferably includes tissue cells that are derived from embryonic, foetal or post-partum tissue. It is preferred that a tissue sample having powerful inductive properties, such as foetal or post-partum organs are used.

Most preferably, the tissue cells are mesenchyme cells. Mesenchyme cells are derived from mesenchymal tissue, which is an embryonic connective tissue, composed of cells contained within an extracellular matrix. Mesenchyme tissue harbors potent inductive signals that act to induce more permissive cell populations to differentiate in a tissue specific manner.

It has been unclear, however, whether any mesenchyme can instruct differentiation of a primative cell line such as an embryonic stem cell, neural stem cell or mesenchymal stem cell line.

Without being limited by theory, it is likely that adult epithelial contain a pluirpotent population of epithelial cells, which might represent the adult stem cell population that has the capacity to give rise to an entirely new organotypic phenotype in response to the inductive and instructive signals from the mesenchyme. However, the idea that the inductive and instructive properties of the mesenchyme are sufficient to direct differentiation of embryonic stem cells was previously unknown and was unexpected.

The tissue cells suitable for use in the methods of the present invention as applied to differentiation of lung are preferably derived from lung mesenchyme, embryonic tissue. The tissue cells may preferably be whole lung tissue sample, lung epithelium and/or mesenchyme as sheets, or lung mesenchyme and/or epithelium in single cell suspensions. In the methods of the present invention, the culturing step may include embedment techniques involving foetal or post-partum tissue samples (either whole tissue sources or parts of tissue, including epithelial or mesenchymal tissues).

It is most preferred that that tissues are selected during organogenesis, preferably when the organ of interest is actually developing. Once the period is identified for the organ, an optimal development period may be determined to select tissue for differentiation of the ES cells. Such optimisation may be conducted by knowing the tissue type and developmental periods and selecting tissues from partitioned time periods. Pseudoglandular and canalicular stage are most preferred as optimal stages for instructing respiratory lineage differentiation in stem cells.

Certain tissue types display organogenesis and undergo this process. Accordingly, it is within the scope of this invention that any tissue that undergoes organogenesis and can be cultured to induce organogenesis in the presence of the stem cells can be used to induce differentiation of the stem cells to a specific cell lineage. It is most preferred that the tissues are selected from the group including, but not limited to, respiratory, pancreatic, mammary, renal, intestinal or hepatic tissues.

The term “extracellular medium” as used herein is taken to mean conditioned medium produced from growing a tissue cell as hereinbefore described in a medium for a period of time so that extracellular factors, such as secreted proteins, produced by the tissue cell are present in the conditioned medium. The medium can include components that encourage the growth of the cells, for example basal medium such as Dulbecco's minimum essential medium, BGJB—Fitton Jackson modified medium, Ham's F12, or foetal calf serum. The extracellular medium may preferably include cellular factors, such as secreted proteins, that are capable of inducing differentiation of a stem cell. Such secreted proteins will typically bind receptors on a cell surface to trigger intracellular pathways which can initiate differentiation of the cell. Examples of suitable extracellular factors include HGF and FGF. The extracellular medium may also contain polar molecules such as steroids which may pass through the cellular and/or nuclear membrane and associate with intracellular factors which trigger a response and initiate differentiation of the cell. Examples of suitable polar molecules include retinoids, glucocorticoids.

The tissue cells and/or the stem cells used in the methods of the present invention may be tagged. Preferably, the stem cells and/or tissue cells used express a transgenic marker protein that allows for identification of differentiated stem cells. Double staining for a reporter gene expressed by stem cells and tissue specific markers may be used to determine the portion of differentiated stem cells relative to the inductive tissue cells in culture. For example, epithelial specific markers such as cytokeratins, mesenchymal markers such as vimentin or lineage specific markers such as surfactant protein C may be used.

In the methods of the present invention the culturing step may involve introducing stem cells to a tissue cell monolayer produced by proliferation of the tissue cells in culture. Preferably, the tissue cell monolayer is grown to confluence and the stem cell is allowed to grow in the presence of extracellular medium of the tissue cells for a period of time sufficient to induce differentiation of the stem cell to a specific cell lineage, wherein the differentiated stem cell is the same cell type as the tissue cells. Alternatively the stem cell is allowed to grow for a period of time sufficient to induce differentiation to an intermediate precursor state in respect to the fully differentiated tissue cell. Alternatively, the stem cell may be allowed to grow in culture containing the extracellular medium of the tissue cell(s), but not in the presence of the tissue cells(s). The tissue cells and stem cells could be separated from each other by a filter or an acellular matrix such as agar.

Suitable conditions for inducing differentiated stem cells are those which are preferably non-permissive for stem cell renewal, but do not kill stem cells or drive them to differentiate exclusively into extraembryonic cell lineages. A gradual withdrawal from optimal conditions for stem cell growth favours differentiation of the stem cell to specific cell types.

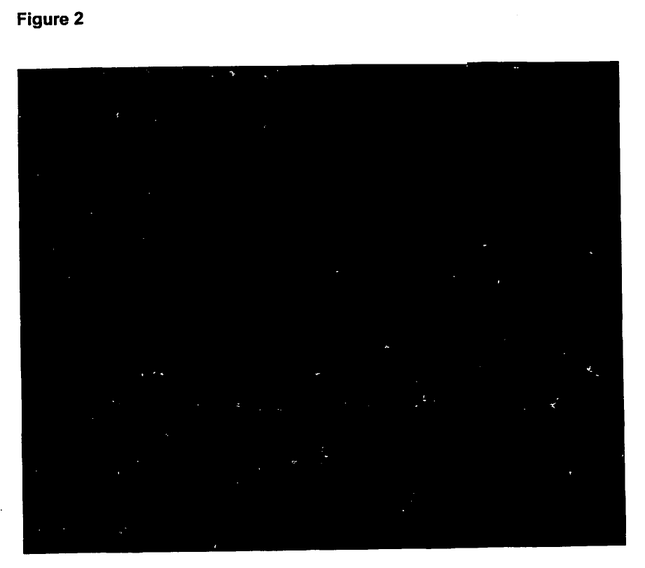

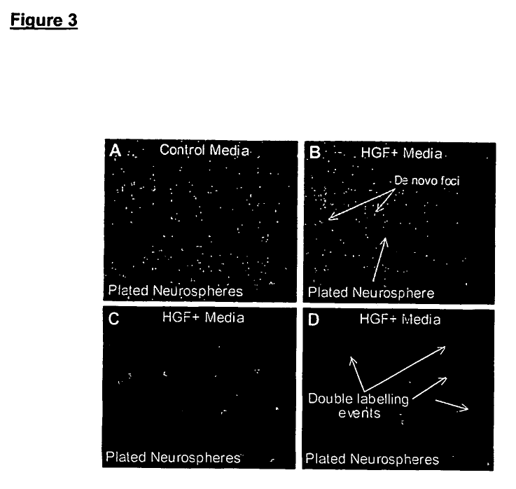

Suitable culture conditions may include the addition of retinoids, glucocorticoids, (as above) or growth factors in co-culture which could increase differentiation rate and/or efficiency. For instance, FIGS. 2 and 3 demonstrate that such growth factors can induce murine ES and neural stem cells to undergo respiratory lineage differentiation in vitro.

Other suitable culturing conditions would include consideration of factors such as cell density. If the tissue cells are plated, then it is preferable that they are grown to confluence. The stem cells may then be preferably dispersed and then introduced to a monolayer of tissue cells. The monolayer is preferably grown to confluence in a suitable medium, such as DMEM or M16 medium. More preferably, the stem cells and tissue cells are co-cultured until a substantial portion of the stem cells have differentiated.

In another aspect of the present invention there is provided a method of inducing differentiation of a stem cell into a specific cell lineage, the method including the steps of:
mixing a first sample of stem cells with a second sample of tissue cells, said cells selected during organogenesis of the tissue to form a cell mixture;
culturing the cell mixture in vitro, under conditions that induce differentiation of a stem cell into a specific cell lineage.

Preferably, the tissue cells are in a substantially single cell suspension prior to mixing with the stem cells. Alternatively, the tissue cells are prepared as a sheet for wrapping the undifferentiated embryoid body. Preferably, the culturing step includes allowing the cell mixture to grow on a permeable membrane, wherein the membrane is in contact with a culture medium, such that the stem cells are induced to differentiate into a specific cell lineage. It is preferred that the permeable membrane be one that may float on the culture medium and that the cell mixture be placed at the air interface. Membranes suitable for such a purpose are millipore or nucleopore filters that preferably have a pore size of less than 0.22 μm.

In another aspect of the present invention there is provided a method of producing differentiated stem cells of a specific cell lineage, the method including:
culturing stem cells in vitro in the presence of a tissue sample and/or extracellular medium of a tissue sample, said sample selected during organogenesis of the tissue, under conditions that induce differentiation of a stem cell into a specific cell lineage; and
recovering differentiated stem cells of a specific cell lineage.

Preferably, the tissue sample is treated to form tissue cells in a substantially single cell suspension prior to culturing with the stem cells. Alternatively, the tissue cells are prepared as a sheet for wrapping an undifferentiated embryoid body.

Pure differentiated stem cells may be recovered by FACS if either the stem cell or the inducing tissue contains a fluorescent marker such as GFP. Alternatively, if the inducing tissue is grown on the opposing surface of a filter to the stem cells, then pure populations of differentiated stem cells may be recovered by mechanical disassociation from the filter.

In a preferred aspect of the present invention there is provided a method of producing differentiated stem cells of a specific cell lineage, the method including:
culturing stem cells in vitro in the presence of tissue cells, said cells selected during organogenesis of the tissue, under conditions that induce differentiation of the stem cell into a specific cell lineage; and
recovering differentiated stem cells of a specific cell lineage.

Preferably, the tissue cells are in a substantially single cell suspension prior to culturing with the stem cells. Alternatively, the tissue cells are prepared as a sheet for wrapping an undifferentiated embryoid body.

The culturing step preferably includes allowing the stem cells to grow on a first surface of a permeable membrane and allowing the tissue cells to grow on an opposite second surface of the permeable membrane, wherein the membrane is in contact with a culture medium, such that the stem cells are induced to differentiate into a specific cell lineage. Differentiated stem cells of a specific cell lineage may then be recovered from the first surface of the permeable membrane. The permeable membrane is preferably, but not limited to a transfilter membrane, where inducing tissue cells and stem cells are placed on opposing sides of the membrane filter.

In the methods of the present invention the stem cells and tissue cells need not be in direct cell-cell contact with one another in culture. The stem cells and tissue cells may be separated by a permeable membrane that allows the diffusion of soluble transmissible signals across the membrane. Suitable permeable membranes may preferably include transfilter membrane, such as millipore or nucleopore filters. The use of a transfilter membrane in the cultures as hereinbefore described provides a convenient and efficient means for obtaining separated and pure populations of induced differentiated stem cells of a specific cell lineage.

In order to facilitate the isolation of pure differentiated stem cells of a specific lineage, heterotypic recombinations of differentiated stem cells and inductive tissue cells as hereinbefore described may be separated by a permeable membrane, such as a nucleopore or millipore filter. Double staining may also be performed to assess the specific cell type of the differentiated stem cell.

In the methods as hereinbefore described, the tissue cells may be derived from embryonic, foetal or post-partum tissue. Preferably, the tissue cells are embryonic mesenchymal cells. More preferably, the tissue cells are derived from lung mesenchyme tissue. The stem cells used in the methods of the present invention are preferably embryonic stem (ES) cells. The tissue cells and/or the stem cells used in the methods of the present invention may be tagged. Preferably, the stem cells used express a transgenic marker protein that allows for identification of differentiated stem cells. The stem cells may be induced to differentiate into specific cell lineages, preferably selected from the group consisting of respiratory, pancreatic, mammary, renal, intestinal, and hepatic.

In the methods of the present invention, the culturing step may preferably include the addition of a growth factor to enhance stem cell differentiation. Suitable growth factors may be preferably selected from epidermal growth factor (EGF), hepatocyte growth factor (HGF) and fibroblast growth factors (FGFs) or steroid hormones (for example, glucocorticoids, vitamin A, thyroid hormone, and retinoids), or other suitable growth enhancing factors such as insulin, serum and cholera toxin. For example, to enhance differentiation of stem cells into cell lineages, growth factors such as FGF may be added to the culture.

In yet another aspect of the invention, there is provided differentiated stem cell of a specific cell lineage produced according to the methods as hereinbefore described. Preferably, the differentiated stem cell is, but not limited to, a lung, kidney, pancreatic, mammary, cardiomyocyte, skeletal muscle cell, , intestinal cell, liver cell, or a haematopoietic cell. The present invention also provides differentiated stem cells produced according to the methods of the invention that may be used for tissue repair, transplantation, cell therapy or gene therapy.

The methods of the present invention also provide a basis for developing cell-based treatments for tissue specific disorders, such as respiratory specific disorders including cystic fibrosis, emphysema, chronic bronchitis, congenital lung hypoplasias and viral infections. For example, stem cells may be co-cultured with lung tissue cells to obtain stem cells differentiated into an intermediate respiratory cell lineage. Intermediate cell lineages would represent any cell type in a stage between derivation from the embryonic inner cell mass, and prior to terminal differentiation of the desired cell type. The intermediately differentiated stem cells may then be propagated to expand numbers. Intermediate cells may be then terminally differentiated in a culture dish for drug discovery programs. Alternatively, the intermediately differentiated stem cells may be transferred to a host (i.e. for example, mouse or human afflicted with a respiratory disease) in a cellular replacement therapy requiring replacement of damaged or sub-optimally functioning respiratory tissue in vivo.

The differentiated cells and their intermediates may be used as a source for isolation or identification of novel gene products including but not limited to growth factors, differentiation factors or factors controlling tissue regeneration, or they may be used for the generation of antibodies against novel epitopes.

The differentiated cells produced according to the methods of the present invention may be clonally expanded. A specific differentiated cell type can be selectively cultivated from a mixture of other cell types and subsequently propagated. Specific differentiated cell types that are clonally expanded can be useful for various applications such as the production of sufficient cells for transplantation therapy, for the production of sufficient RNA for gene discovery studies etc. The differentiated cells may be used to establish cell lines according to conventional methods.

The differentiated cells produced according to the methods of the present invention may be genetically modified. For instance, a genetic construct may be inserted to a differentiated cell at any stage of cultivation. The genetically modified cell may be used after transplantation to carry and express genes in target organs in the course of gene therapy.

The differentiated stem cells produced according to the methods of the present invention may be preserved or maintained by any methods suitable for storage of biological material. Effective preservation of differentiated cells is highly important as it allows for continued storage of the cells for multiple future usage. Traditional slow freezing methods, commonly utilised for the cryo-preservation of cell lines, may be used to cryo-preserve differentiated cells.

The present invention further provides a cell composition including a differentiated cell produced by the method of the present invention, and a carrier. The carrier may be any physiologically acceptable carrier that maintains the cells. It may be PBS or other minimum essential medium known to those skilled in the field. The cell composition of the present invention can be used for biological analysis or medical purposes, such as transplantation. In addition, the cell composition of the present invention can be used in methods of treating diseases or conditions, such as respiratory disease.

The present invention will now be more fully described with reference to the accompanying examples and drawings. It should be understood, however that the description following is illustrative only and should not be taken in any way as a restriction on the generality of the invention described above.

### EXAMPLES

### Example 1

### Differentiation of Embryonic Stem Cells into Lung Aggregates

Between 4 and 8 foetal mouse lungs were collected on either embryonic day 11.5 (E11.5), E12.5 or E13.5 in PBS and transferred to 0.25% trypsin/ 0.04% EDTA for mechanical dissociation into single cell suspension by aspiration using a p200 Gilson pipette and pipette tip. Trypsin was deactivated by washing in DMEM (GIBCO) containing 10% foetal calf serum (FCS). Cells and medium were transferred to a 1.5 ml eppendorf tube for centrifugation at 300 g for four minutes. The supernatant was decanted and the cell pellet resuspended in 300 μl of BGJB Fitton Jackson modified media (GIBCO) containing 150 μg/ml ascorbic acid and supplemented with 5% FCS and 2 mM L-glutamine (hereafter referred to as culture media).

Between 20,000 or 10,000 ZIN40 or green fluorescent protein (GFP) embryonic stem (ES) cells were then mixed gently with the 300 μl embryonic lung cell suspension. The cell mixture was then centrifuged again for 4 minutes at 300 g and resuspended in 1 μl of culture media. This 1 μl of concentrated cell suspension was then transferred onto a millipore filter floating on pre-equilibrated culture media using a finely drawn glass pipette and incubated at 37° C. in 5% CO2. Culture media was changed every 2 days. After the designated culture period, embryonic lung/ES cell aggregates were peeled from the millipore filters with forceps and transferred to a 2 ml round bottom eppendorf tube containing phosphate buffered saline (PBS). Aggregates were washed in PBS 3 times to remove culture media and fixed for 1 hour at 4° C. in 4% paraformaldehyde on a rocking stage. Aggregates were then once again washed in PBS 3 times to remove fixative and processed for cellular analysis.

### Example 2

### Differentiation of Neural Stem Cells into a Respiratory Lineage

Neurospheres derived in culture from green fluorescent protein (GFP) transgenic foetal mouse brains were analysed for the expression of the neural stem (NS) cell markers nestin and musashi. Zin 40 ES cells and EBs were cultured as previously described (Munsie et al., 2000). Embryos were recovered from E12.5 EGFP +/− mouse matings (Jackson laboratories) and EGFP positive neurospheres generated according to Reynolds and Weiss (1992).

After four days of culture, either 50 embryoid bodies or 50 neurospheres were collected and placed in a 35 mm culture dish in DMEM. Either HGF or NGF was added at a final concentration of 20 ng/ml and 100 ng/ml to the stem cells for eight days following plating in 35 mm dishes. In an alternative and separate treatment, the embryoid bodies or neurospheres were treated with DMEM supplemented with HGF+ media (DMEM containing 3% charcoal stripped foetal calf serum, 10 μg/ml insulin, 1 μg/ml cholera toxin, 25 ng/ml epidermal growth factor, 10 ng/ml hepatocyte growth factor and 25 ng/ml FGF7). Cells were stained according to standard procedures.

Results

Aggregates of foetal mouse lung induced immunoreactivity to the respiratory-specific marker SPC of both mouse and human ES cells. Of all mouse ES cell derivatives cultured as aggregates, 28% demonstrated immunoreactivity to the SPC-specific antibody. Although only few human ES cell derivatives cultured as aggregates demonstrated immunoreactivity to the SPC-specific antibody, culture conditions remain to be optimised and exact numbers undergoing respiratory-specific differentiation remain to be determined. Both mouse and human SPC immunoreactive ES cell derivatives in aggregates incorporated as tubule structures reminiscent of functional respiratory tubules. Tubules were composed either solely of ES cell derivatives or of a mixture of contaminating endogenous respiratory epithelia and ES cell derivatives. Furthermore, ES cell derivative SPC localisation was found to be polarised to the apical surface, indicative of normal functional respiratory cell types.

Both mouse and human ES cells cultured alone as embryoid bodies differentiated into derivatives of three germ layers (mesoderm, ectoderm and endoderm; data not shown), thus evidencing their ability to form all tissues of the body. However, we are as yet to identify SPC immunoreactive ES cell derivatives in these aggregates at any significant level. Because our aggregate system results in the induction of mouse ES cell derivative SPC immunoreactivity at an incidence of 28%, our aggregate system can thus be said to direct differentiation of both ES cells.

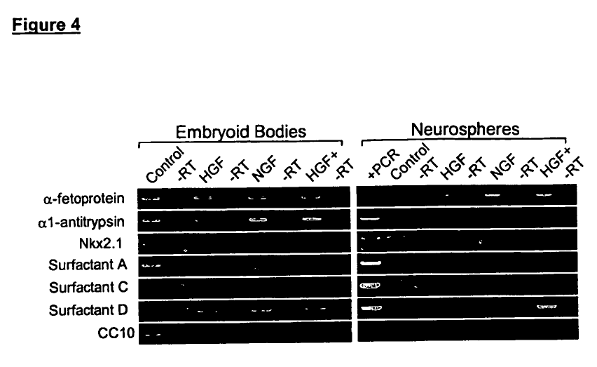

The morphology of EBs and neurospheres plated at high density in either HGF or NGF was similar to that of EBs and neurospheres cultured in control media after 8 days (data not shown). Neurospheres cultured in HGF+ media demonstrated a relatively reduced propensity to plate down as a monolayer when compared to controls, instead forming de novo foci within the dish (FIGS. 3A,B). Double Hoechst staining (nuclear) and anti-surfactant C immunocytochemistry (cytoplasmic) demonstrated that many of these foci contained surfactant C Ab reactive cells (FIGS. 3C,D). Following culture at high density, RT-PCR demonstrated amplification of α-fetoprotein (an endoderm-specific marker) and Nkx2.1 (an early marker of lung, thyroid, pituitary and diencephelon development) transcripts in all culture conditions (FIG. 4). Semi-quantitative PCR demonstrated upregulation of Surfactant A transcription within neurospheres cultured in the presence of either NGF or HGF+ media. Similarly, upregulation of Surfactant C transcription was observed in both EBs and neurospheres cultured in the presence of HGF+ medium, whereas Surfactant D transcription was upregulated by treatment with HGF, NGF or HGF+ medium. Although α1-antitrypsin and clara cell secretory protein (CC10) transcripts were detected in all EB culture conditions, they were not detected in neurospheres under the culture conditions tested. These studies demonstrate that in vitro, NS and ES cells can be induced to express marker representatives of fully differentiated respiratory lineages.

Throughout the description and claims of this specification, the word “comprise” and variations of the word, such as “comprising” and “comprises”, is not intended to exclude other additives, components, integers or steps.

The discussion of prior art documents, acts, devices and the like is included in this specification solely for the purpose of providing a context for the present invention. It is not suggested or represented that any or all of these matters formed part of the prior art base or were common general knowledge in the field relevant to the present invention as it existed in Australia before the filing date of this application.

Finally, the invention as hereinbefore described is susceptible to variations, modifications and/or additions other than those specifically described and it is understood that the invention includes all such variations, modifications and/or additions which may be made it is to be understood that various other modifications and/or additions which fall within the scope of the description as hereinbefore described.

### REFERENCES

Reubinoff BE, Pera MF, Fong CY, Trounson A, Bongso A. (2000) Embryonic stem cell lines from human blastocysts: somatic differentiation in vitro. Nat Biotechnol. 18(4):399-404.

1-35. (canceled)

36. A method of inducing differentiation of a stem cell into a specific cell lineage, comprising:
culturing a stem cell in vitro under conditions that induce differentiation of the stem cell into a specific cell lineage, said step of culturing taking place in the presence of at least one of
(i) a tissue sample from a tissue, and
(ii) extracellular medium from a tissue sample from a tissue,
wherein said tissue sample is selected during organogenesis of the tissue.

37. A method of treating a tissue specific disorder, comprising:
(a) obtaining a differentiated stem cell or a product of a differentiated stem cell according to a method that comprises culturing a stem cell in vitro under conditions that induce differentiation of the stem cell into a specific cell lineage, said step of culturing taking place in the presence of at least one of
(i) a tissue sample from a tissue, and
(ii) extracellular medium from a tissue sample from a tissue,
wherein said tissue sample is selected during organogenesis of the tissue,
and wherein said differentiated stem cell corresponds to a tissue of a tissue specific disorder; and
(b) administering the differentiated stem cell to a corresponding tissue of the tissue specific disorder in a patient in need.

38. The method of claim 37 wherein the differentiated stem cell is selected from the group consisting of a lung cell, a kidney cell, a pancreatic cell, a mammary cell, a cardiomyocyte, an intestinal cell, a hepatic cell, a brain cell, a muscle cell and a reproductive cell.

39. The method of claim 37 wherein the differentiated stem cell is from a lung cell lineage.

40. The method of claim 37 wherein the differentiated stem cell is genetically modified.

41. A method of treating a tissue specific disorder, comprising:
obtaining an intermediate differentiated stem cell or a product of an intermediate differentiated stem cell according to a method that comprises culturing a stem cell in vitro under conditions that induce differentiation of the stem cell into a specific cell lineage to produce said intermediate differentiated stem cell, said step of culturing taking place in the presence of at least one of
(i) a tissue sample from a tissue, and
(ii) extracellular medium from a tissue sample from a tissue,
wherein said tissue sample is selected during organogenesis of the tissue,
wherein said intermediate differentiated stem cell is partially differentiated from an embryonic inner cell mass stage to a terminally differentiated stage,
and wherein said stem cell corresponds to a tissue of the tissue specific disorder; and
administering the differentiated stem cell to the corresponding tissue of the tissue specific disorder in a patient in need.

42. The method of claim 41 wherein the intermediate differentiated stem cell is selected from the group consisting of a lung cell, a kidney cell, a pancreatic cell, a mammary cell, a cardiomyocyte, an intestinal cell, a hepatic cell, a brain cell, a muscle cell and a reproductive cell.

43. The method of claim 41 wherein the intermediate differentiated stem cell is from a lung cell lineage.

44. The method of claim 41 wherein the intermediate differentiated stem cell is genetically modified.

45. The method of any one of claims 36, 37 or 41 wherein the stem cell is selected from the group consisting of an embryonic stem cell, a pluripotent stem cell, a haematopoietic stem cell, a totipotent stem cell, a mesenchymal stem cell, a neural stem cell, an adult stem cell and a genetically modified stem cell, wherein the genetically modified stem cell is selected from the group consisting of an embryonic stem cell, a pluripotent stem cell, a haematopoietic stem cell, a totipotent stem cell, a mesenchymal stem cell, a neural stem cell and an adult stem cell.

46. The method of any one of claims 36, 37 or 41 wherein the stem cell is derived from an embryonic cell line or an embryonic tissue, or by somatic nuclear transfer.

47. The method of any one of claims 36, 37 or 41 wherein the stem cell comprises an embryonic stem cell.

48. The method of claim 47 wherein the embryonic stem cell comprises a human embryonic stem cell or a mouse embryonic stem cell.

49. The method of any one of claims 36, 37 or 41 wherein the stem cell comprises a human embryonic stem cell.

50. The method of any one of claims 36, 37 or 41 wherein the tissue sample and the stem cell express similar markers.

51. The method of any one of claims 36, 37 or 41 wherein the tissue sample comprises a single cell suspension.

52. The method of any one of claims 36, 37 or 41 wherein the tissue sample is selected at a pseudoglandular or canalicular stage of development during organogenesis.

53. The method of any one of claims 36, 37 or 41 wherein the tissue sample comprises a tissue cell type selected from the group consisting of an epithelial tissue cell, an endothelial connective tissue cell, a skeletal tissue cell, a muscle tissue cell, a glandular tissue cell, and a nervous tissue cell.

54. The method of claim 53 wherein the tissue cell type comprises a mesenchymal cell.

55. The method of any one of claims 36, 37 or 41 wherein the tissue sample is derived from an organ selected from the group consisting of a respiratory organ, prostate gland, a reproductive organ, kidney, pancreas, mammary gland, brain, heart, muscle, liver, and intestine.

56. The method of any one of claims 36, 37 or 41 wherein the tissue sample comprises a mixture of mesenchymal tissue and epithelial tissue.

57. The method of claim 56 wherein the tissue sample comprises fetal mesenchymal tissue or a mixture of fetal mesenchymal and epithelial tissue

58. The method of any one of claims 36, 37 or 41 wherein the specific cell lineage is selected from the group consisting of respiratory cell lineage, pancreatic cell lineage, mammary cell lineage, renal cell lineage, intestinal cell lineage, hepatic cell lineage, neural cell lineage, cardiac cell lineage, and muscle cell lineage.

59. The method of any one of claims 36, 37 or 41 wherein the specific cell lineage is a respiratory cell lineage.

60. The method of any one of claims 36, 37 or 41 wherein the stem cell and the tissue sample are co-cultured but are not in direct cell-cell contact.

61. The method according to claim 60 wherein the stem cell and the tissue sample are separated by a permeable membrane.

62. The method according to claim 61 wherein the permeable membrane is a millipore filter or a nucleopore filter.

63. The method of either claim 37 or claim 41 wherein the tissue specific disorder is selected from the group consisting of cystic fibrosis, emphysema, chronic bronchitis, congenital lung hypoplasia and viral infection.

64. A differentiated stem cell of a specific stem cell lineage that is prepared by a method which comprises culturing a stem cell in vitro under conditions that induce differentiation of the stem cell into a specific cell lineage, said step of culturing taking place in the presence of at least one of (i) a tissue sample from a tissue, and (ii) extracellular medium from a tissue sample from a tissue, wherein said tissue sample is selected during organogenesis of the tissue.

65. A differentiated stem cell according to claim 64 that is selected from the group consisting of a lung cell, a kidney cell, a pancreatic cell, a mammary cell, a cardiomyocyte, an intestinal cell, a hepatic cell, a brain cell, a muscle cell and a reproductive cell.

66. A differentiated stem cell according to claim 64 that is from a lung cell lineage.

67. A differentiated stem cell according to claim 64 that is genetically modified.

68. A cell composition comprising a differentiated stem cell according to any one of claims 64-66.

69. An intermediate differentiated stem cell of a specific stem cell lineage prepared by a method which comprises culturing a stem cell in vitro under conditions that induce differentiation of the stem cell into a specific cell lineage to produce said intermediate differentiated stem cell, said step of culturing taking place in the presence of at least one of (i) a tissue sample from a tissue, and (ii) extracellular medium from a tissue sample from a tissue, wherein said tissue sample is selected during organogenesis of the tissue, and wherein said intermediate differentiated stem cell is partially differentiated from an embryonic inner cell mass stage to a terminally differentiated stage.

70. An intermediate differentiated stem cell according to claim 69 that is selected from the group consisting of a lung cell, a kidney cell, a pancreatic cell, a mammary cell, a cardiomyocyte, an intestinal cell, a hepatic cell, a brain cell, a muscle cell and a reproductive cell.

71. An intermediate differentiated stem cell according to claim 69 which is from a lung cell lineage.

72. An intermediate differentiated stem cell according to claim 69 which is genetically modified.

73. A cell composition comprising an intermediate differentiated stem cell according to any one of claims 69-72.
